(19) United States
(12) Patent Application Publication
(10) Pub. No.: US 2025/0336067 A1
(43) Pub. Date: Oct. 30, 2025

(54) METHOD AND SYSTEM FOR DETECTING MALPLACEMENT AND MALPOSITIONING OF MEDICAL LINES AND TUBES

(71) Applicant: Cloudphysician Healthcare Pvt Ltd, Bangalore (IN)

(72) Inventors: Carl Britto, Bengaluru (IN); Dhruv Sud, Bengaluru (IN)

(21) Appl. No.: 19/193,896

(22) Filed: Apr. 29, 2025

(30) Foreign Application Priority Data
Apr. 30, 2024 (IN) 202441034483

(51) Int. Cl. G06T 7/00; A61M 39/08; G06T 7/70; G06V 10/764; G06V 10/774; G06V 10/778; G06V 20/50; G16H 10/60; G16H 40/20

(52) U.S. Cl. CPC G06T 7/0012; A61M 39/08; G06T 7/70; G06V 10/764; G06V 10/774; G06V 10/7792; G06V 20/50; G16H 10/60; G16H 40/20; A61M 2205/13; A61M 2205/18; A61M 2205/3306; G06T 2207/20081; G06T 2207/30004; G06V 2201/03

(57) ABSTRACT

This disclosure relates to a method and system for detecting malplacement and malpositioning of medical lines and tubes. The method may include receiving real-time image data corresponding to a patient from one or more cameras and patient data from an Electronic Medical Record (EMR) of the patient. At least one medical line or tube may be at least partially inserted in at least one body part of the patient. The method may further include determining a set of optimal parameters for each of the at least one medical line or tube with respect to a corresponding body part of the patient based on the patient data. The method may further include detecting malplacement and malpositioning of the at least one medical line or tube based on the real-time image data, the set of optimal parameters, and predefined insertion criteria using a computer vision-based Machine Learning (ML) model.

### TECHNICAL FIELD

This disclosure relates generally to the field of healthcare, and more particularly to method and system for detecting malplacement and malpositioning of medical lines and tubes via machine learning, specifically computer vision.

### BACKGROUND

Insertion and placement of medical lines and tubes into patient body parts are routine procedures conducted in various clinical settings, including hospitals, ambulatory care centers, and emergency rooms. Numerous peripheral lines, central lines, chest tubes, endotracheal (ET) tubes, surgical site drains, and catheters are inserted in patients for access (i.e., patient intake) and drainage (i.e., patient output) in critical settings on a daily basis.

While these procedures are crucial for delivering and draining fluids, to and from the patients, there are complications which can often arise, which are sometimes peri-procedural but may also arise late in Intensive Care Unit (ICU) stay. Some examples where peri-procedural interventions may be required are wrong size of an Intravenous (IV) cannula, wrong type of laryngoscope blade, wrong size of the ET tube, wrong site of placement (i.e., malplacement and malpositioning), etc. Further, once a medical line/drain is in place, there may be issues with how deep it is (in mm) placed into insertion site of the patient. Some additional complications may be thrombophlebitis, extravasation, and blockages in the medical line/drain. Failure to detect tube malfunction or tube malposition can lead to clinical deterioration and even death Interventions for tube malposition range from simple adjustment to total tube replacement.

Currently, healthcare professionals rely on daily monitoring via clinical assessment, anatomical landmarks, and imaging techniques, such as X-rays, fluoroscopy, and ultrasound, to verify the placement and positioning of the medical lines and tubes. However, these methods often involve delays, increased radiation exposure, and resource-intensive processes and are not suited for 24×7 real-time monitoring.

Therefore, there is a need for an improved, automated method and system that can accurately and reliably detect malplacement and malpositioning of medical lines and tubes in real-time, thus mitigating risks associated with these critical procedures.

### SUMMARY

In one embodiment, a method for detecting malplacement and malpositioning of medical lines or tubes may be disclosed. In one example, the method may include receiving real-time image data corresponding to a patient from one or more cameras and patient data from an Electronic Medical Record (EMR) of the patient. At least one medical line or tube may be at least partially inserted in at least one body part of the patient. The real-time image data may include a plurality of images capturing the at least one medical line or tube and the corresponding at least one body part. The method may further include determining a set of optimal parameters for each of the at least one medical line or tube with respect to a corresponding body part of the patient based on the patient data. The method may further include detecting malplacement and malpositioning of the at least one medical line or tube based on the real-time image data, the set of optimal parameters, and predefined insertion criteria using a Machine Learning (ML) model.

In one embodiment, a system for detecting malplacement and malpositioning of medical lines or tubes may be disclosed. In one example, the system may include a processor and a memory communicatively coupled to the processor. The memory may store processor-executable instructions, which, on execution, may cause the processor to receive real-time image data corresponding to a patient from one or more cameras and patient data from an Electronic Medical Record (EMR) of the patient. At least one medical line or tube may be at least partially inserted in at least one body part of the patient. The real-time image data may include a plurality of images capturing the at least one medical line or tube and the corresponding at least one body part. The processor-executable instructions, on execution, may further cause the processor to determine a set of optimal parameters for each of the at least one medical line or tube with respect to a corresponding body part of the patient based on the patient data. The processor-executable instructions, on execution, may further cause the processor to detect malplacement and malpositioning of the at least one medical line or tube based on the real-time image data, the set of optimal parameters, and predefined insertion criteria using a Machine Learning (ML) model.

In one embodiment, a non-transitory computer-readable medium storing computer-executable instructions for detecting malplacement and malpositioning of medical lines or tubes may be disclosed. In one example, the stored instructions, when executed by a processor, may cause the processor to perform operations including receiving real-time image data corresponding to a patient from one or more cameras and patient data from an Electronic Medical Record (EMR) of the patient. At least one medical line or tube may be at least partially inserted in at least one body part of the patient. The real-time image data may include a plurality of images capturing the at least one medical line or tube and the corresponding at least one body part. The operations may further include determining a set of optimal parameters for each of the at least one medical line or tube with respect to a corresponding body part of the patient based on the patient data. The operations may further include detecting malplacement and malpositioning of the at least one medical line or tube based on the real-time image data, the set of optimal parameters, and predefined insertion criteria using a Machine Learning (ML) model.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for detecting malplacement and malpositioning of medical lines or tubes, in accordance with some embodiments.

FIG. 2 illustrates a functional block diagram of an exemplary system for detecting malplacement and malpositioning of medical lines or tubes, in accordance with some embodiments.

FIG. 3 illustrates a flowchart of an exemplary process for detecting malplacement and malpositioning of medical lines and tubes, in accordance with some embodiments.

FIG. 4 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

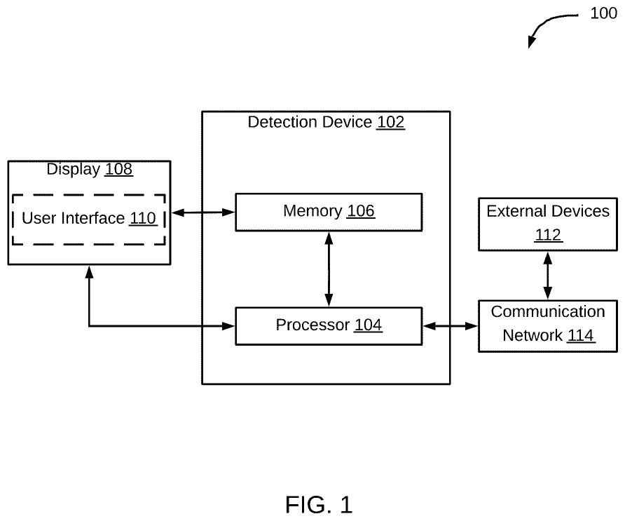

Referring now to FIG. 1, an exemplary system 100 for detecting malplacement and malpositioning of medical lines and tubes is illustrated, in accordance with some embodiments. The system 100 may implement a detection device 102 (for example, server, desktop, laptop, notebook, netbook, tablet, smartphone, mobile phone, or any other computing device), in accordance with some embodiments of the present disclosure. The detection device 102 may detect malplacement and malpositioning of medical lines and tubes using a Machine Learning (ML) model.

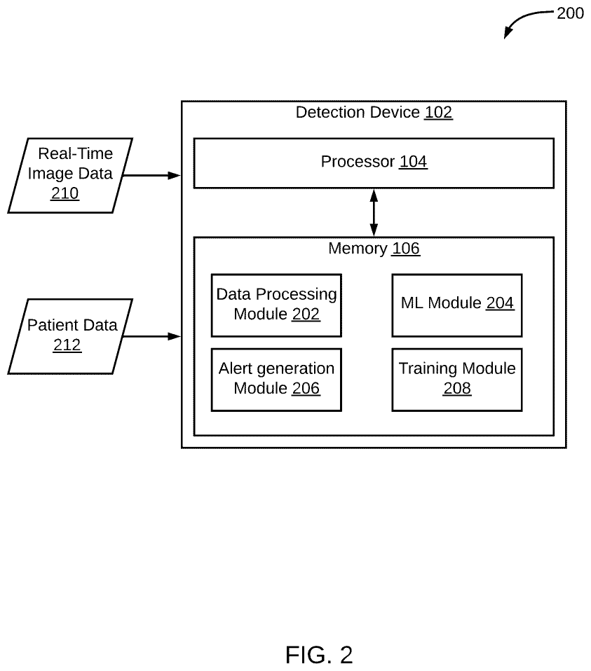

As will be described in greater detail in conjunction with FIGS. 2-4, the detection device 102 may receive real-time image data corresponding to a patient from one or more cameras and patient data from an Electronic Medical Record (EMR) of the patient. At least one medical line or tube is at least partially inserted in at least one body part of the patient. The real-time image data may include a plurality of images capturing the at least one medical line or tube and the corresponding at least one body part. Further, the detection device 102 may determine a set of optimal parameters for each of the at least one medical line or tube with respect to a corresponding body part of the patient based on the patient data. Further, the detection device 102 may detect malplacement and malpositioning of the at least one medical line or tube based on the real-time image data, the set of optimal parameters, and predefined insertion criteria using an ML model.

In some embodiments, the detection device 102 may include one or more processors 104 and a memory 106. Further, the memory 106 may store instructions that, when executed by the one or more processors 104, cause the one or more processors 104 to detect malplacement and malpositioning of medical lines and tubes. The memory 106 may also store various data (for example, patient data, real-time image data, training dataset, ML model parameters, and the like) that may be captured, processed, and/or required by the system 100.

The system 100 may further include a display 108. The system 100 may interact with a user via a user interface 110 accessible via the display 108. The system 100 may also include one or more external devices 112. In some embodiments, the detection device 102 may interact with the one or more external devices 112 over a communication network 114 for sending or receiving various data. The external devices 112 may include, but may not be limited to, a remote server, a digital device, one or more cameras, or another computing system.

Referring now to FIG. 2, a functional block diagram of an exemplary system 200 for detecting malplacement and malpositioning of medical lines and tubes is illustrated, in accordance with some embodiments. Malplacement and malpositioning may be defined as an incorrect or wrong placement of the medical lines and tubes with respect to body of the patient. Some examples of incorrect placement of the medical lines and tubes may be a tube inserted at an incorrect site (i.e., body part), a tube inserted at wrong depth (too deep into the site or not deep enough), a tube inserted at a wrong alignment (malalignment), a tube of wrong size, wrong type of tube used, malfunctioning of a tube, etc. The detection device 102 may include the processor 104 and the memory 106 communicatively coupled with each other. The memory 106 may include a data processing module 202, an ML module 204, an alert generation module 206, and a training module 208.

The data processing module 202 may receive real-time image data 210 corresponding to a patient from one or more cameras and patient data 212 from an EMR of the patient. It should be noted that a medical line or tube is at least partially inserted in at least one body part of the patient. In an embodiment, the medical line or tube is in process of being inserted into a body part of the patient. In another embodiment, the medical line or tube is completely inserted into a body part of the patient. By way of an example, the medical line or tube may include, but may not be limited to peripheral lines, central lines, chest tubes, Endotracheal (ET) tubes, surgical site drains, catheters, leads, Intravenous (IV) cannula, nasal cannula, and the like. The patient data 212 may include physiological data of the patient and procedure orders obtained from the EMR.

The patient may be in a room (for example, a hospital ward, an Intensive Care Unit (ICU), an emergency ward, a private room at hospital or at home, etc.). The one or more cameras may be positioned in the room so as to capture the patient and the medical line or tube from one or more angles. The one or more cameras may be standard cameras (such as, Closed Circuit Television (CCTV) cameras, digital cameras, smartphone cameras, etc.). The one or more cameras may be configured to continuously (or periodically) capture real-time images of the patient and the medical line or tube. Alternatively, the one or more cameras may be configured to continuously record/stream video corresponding to the patient and the medical line or tube. In an embodiment, a first camera may capture real-time image data corresponding to the patient and a second camera may capture real-time image data corresponding to the medical line or tube. Thus, the real-time image data 210 may include a plurality of images capturing the medical line or tube and the corresponding at least one body part. By way of an example, each of the plurality of images of the real-time image data 210 may be a regular light image, an Infrared (IR) light image, a video frame, or the like.

Further, the data processing module 202 may preprocess the real-time image data 210 and the patient data 212 using one or more preprocessing techniques. The data processing module 202 may determine a set of optimal parameters for each of the medical line or tube with respect to a corresponding body part of the patient based on the patient data 212. The set of optimal parameters may include optimal sizing parameters of the medical line or tube. Thus, using the physiological data of the patient and the procedure orders, an optimal size of the medical line or tube (e.g., IV cannula, ET tube, etc.) with respect to the corresponding body part of the patient may be determined. For example, the optimal size may be defined in terms of an optimal length of the medical line or tube and/or an optimal thickness of the medical line or tube.

The ML module 204 may include an ML model (for example, a deep learning model (such as a Convolutional Neural Network (CNN) model) or a computer vision model (such as a You Only Look Once (YOLO) model)). Further, the ML module 204 may detect malplacement and malpositioning of the medical line or tube based on the real-time image data 210, the set of optimal parameters, and predefined insertion criteria using the ML model.

To detect the malplacement and malpositioning, the ML module 204 may identify the medical line or tube in an image via object detection and boundary detection techniques using the ML model. Further, the ML module 204 may identify a site of contact of each of the identified medical line or tube with the corresponding body part of the patient using the ML model. Further, the ML module 204 may determine a set of current parameter values based on each of the identified medical line or tube and the identified site of contact. Further, the ML module 204 may compare the set of current parameter values with the corresponding set of optimal parameter values. Further, the ML module 204 may detect the malplacement and malpositioning based on the comparison and the predefined insertion criteria.

In an embodiment, the set of current parameter values may include size parameters, contact parameters, and a type of the medical line or tube. In such an embodiment, the set of optimal parameters may include optimal size parameters (including the determined optimal size). The predefined insertion criteria may include an optimal site of insertion of the medical line or tube into the body part of the patient and an optimal type of the medical line or tube to be used for the body part. For example, the optimal site of insertion of an arterial line (A-line) may be one of a femoral, a brachial, a radial, or a dorsalis pedis artery. The size parameters may then be compared with the optimal size parameters. The contact parameters may be compared with the predefined insertion criteria. Also, the type of the medical line or tube may be compared with the optimal types of the medical line or tube. If the size parameters differ from the optimal size parameters beyond a predefined size threshold, the ML module 204 may detect the malplacement and malpositioning of the medical line or tube. Similarly, if the contact parameters differ from the predefined insertion criteria beyond a predefined contact threshold or if the type of the medical line or tube differs from the optimal type, the ML module 204 may detect the malplacement and malpositioning of the medical line or tube.

It should be noted that for ease of explanation, the system 200 is described as detecting the malplacement and malpositioning of a single medical line or tube. However, the same logic may be applied for any number of medical lines or tubes inserted into the body of the patient. In other words, the detection device 102 may be used to detect malplacement and malpositioning of each of a plurality of lines or tubes inserted into one or more body parts of the patient.

In some embodiments, the ML module 204 may generate a recommendation corresponding to the detected malplacement and malpositioning. The recommendation may be indicative of one or more solutions to the detected malplacement and malpositioning. For example, the recommendation for a tube malposition may be “replace the tube”, the recommendation for a wrongly sized tube may be “use a smaller sized tube”, or “use an appropriate sized tube” etc. In an embodiment, the recommendation may be generated using a Large Language Model (LLM) such as, but not limited to, Generative Pre-Trained Transformer (GPT), Pathways Language Model (PaLM), Gemini, Grok, Large Language Model Meta AI (LLaMA), or the like. In such an embodiment, the recommendation may be in natural language.

In some embodiments, the ML module 204 may also classify the medical line or tube into a patient intake line or a patient output line using the ML model. The training module 208 may train the ML model based on a training dataset using supervised learning techniques. The training dataset may include training data corresponding to correctly placed medical lines and tubes and incorrectly placed medical lines and tubes. In an embodiment, the training data may be labelled data.

By way of an example, the ML module 204 may use computer vision techniques on regular light images, IR light images, and video to detect blood or other blockages in a tube inserted in the body. Failure to detect such kind of tube malfunction or tube malposition can lead to clinical deterioration and even death of the patient. Additionally, the computer vision techniques may be used to detect depth measurements of the tube. A combination of both regular light images and IR light images may allow the detection device 102 with object detection, boundary detection, removing background noise for accurate assessment and recommendations.

Upon detecting the malplacement and malpositioning, the alert generation module 206 may generate an alert for a medical care supervisor through the EMR. In an embodiment, the alert may be generated through a tele-ICU system. In an embodiment, the alert may be rendered in the form of a notification to a user device associated with the medical care supervisor. Thus, the detection device 102 helps in determination of optimal sizing and detection of malplacement of medical lines and tubes.

It should be noted that all such aforementioned modules 202-208 may be represented as a single module or a combination of different modules. Further, as will be appreciated by those skilled in the art, each of the modules 202-208 may reside, in whole or in parts, on one device or multiple devices in communication with each other. In some embodiments, each of the modules 202-208 may be implemented as a dedicated hardware circuit comprising custom application-specific integrated circuit (ASIC) or gate arrays, off-the-shelf semiconductors such as logic chips, transistors, or other discrete components. Each of the modules 202-208 may also be implemented in a programmable hardware device such as a field programmable gate array (FPGA), programmable array logic, programmable logic device, and so forth. Alternatively, each of the modules 202-208 may be implemented in software for execution by various types of processors (e.g., processor 104). An identified module of executable code may, for instance, include one or more physical or logical blocks of computer instructions, which may, for instance, be organized as an object, procedure, function, or other construct. Nevertheless, the executables of an identified module or component need not be physically located together, but may include disparate instructions stored in different locations which, when joined logically together, include the module, and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for detecting malplacement and malpositioning of medical lines and tubes. For example, the exemplary system 100 and the associated detection device 102 may detect the malplacement and malpositioning of medical lines and tubes by the processes discussed herein. In particular, as will be appreciated by those of ordinary skill in the art, control logic and/or automated routines for performing the techniques and steps described herein may be implemented by the system 100 and the detection device 102 either by hardware, software, or combinations of hardware and software. For example, suitable code may be accessed and executed by the one or more processors 104 on the system 100 to perform some or all of the techniques described herein. Similarly, application specific integrated circuits (ASICs) configured to perform some or all of the processes described herein may be included in the one or more processors 104 on the system 100.

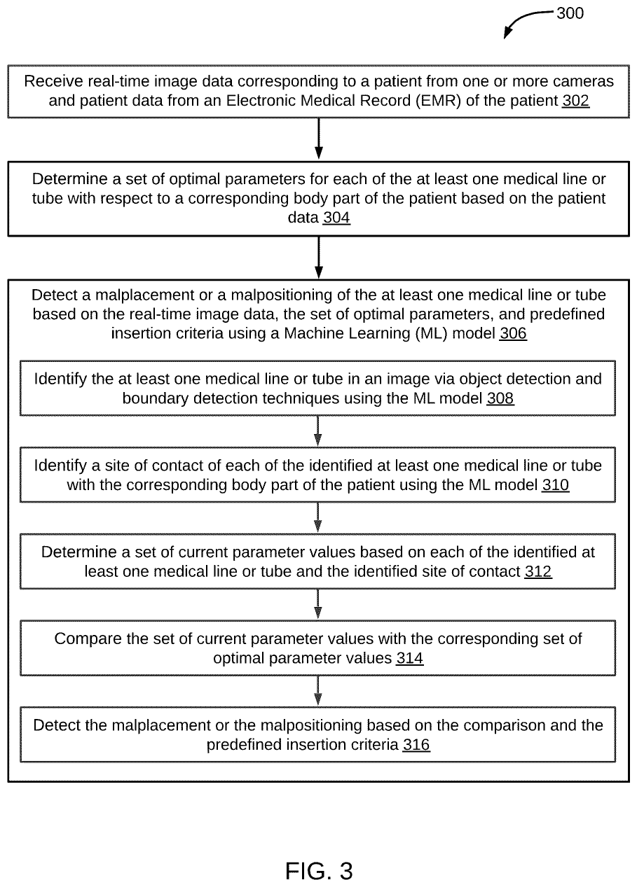

Referring now to FIG. 3, an exemplary process 300 for detecting malplacement and malpositioning of medical lines and tubes is depicted via a flowchart, in accordance with some embodiments. In an embodiment, the process 300 may be implemented by the detection device 102 of the system 100. FIG. 3 is explained in conjunction with the FIGS. 1 and 2.

The process 300 includes receiving, by the data processing module 202, real-time image data (such as the real-time image data 210) corresponding to a patient from one or more cameras and patient data (such as the patient data 212) from an EMR of the patient, at step 302. At least one medical line or tube is at least partially inserted in at least one body part of the patient. The real-time image data may include a plurality of images capturing the at least one medical line or tube and the corresponding at least one body part. By way of an example, each of the plurality of images of the real-time image data may be one of a regular light image, an IR light image, a video frame, or the like.

Further, the process 300 includes determining, by the data processing module 202, a set of optimal parameters for each of the at least one medical line or tube with respect to a corresponding body part of the patient based on the patient data, at step 304.

Further, the process 300 includes detecting, by the ML module 204, a malplacement and malpositioning of the at least one medical line or tube based on the real-time image data, the set of optimal parameters, and predefined insertion criteria using an ML model, at step 306.

The step 306 of the process 300 may include identifying, by the ML module 204, the at least one medical line or tube in an image via object detection and boundary detection techniques using the ML model, at step 308. Further, the step 306 of the process 300 may include identifying, by the ML module 204, a site of contact of each of the identified at least one medical line or tube with the corresponding body part of the patient using the ML model, at step 310. Further, the step 306 of the process 300 may include determining, by the ML module 204, a set of current parameter values based on each of the identified at least one medical line or tube and the identified site of contact, at step 312. Further, the step 306 of the process 300 may include comparing, by the ML module 204, the set of current parameter values with the corresponding set of optimal parameter values, at step 314. Further, the step 306 of the process 300 may include detecting, by the ML module 204, the malplacement and malpositioning based on the comparison and the predefined insertion criteria, at step 316.

The process 300 may include classifying, by the ML module 204, the at least one medical line or tube into a patient intake line or a patient output line using the ML model. In some embodiments, the process 300 may include training, by the training module 208, the ML model based on a training dataset using supervised learning techniques.

In some embodiments, the process 300 may include upon detecting the malplacement and malpositioning, generating, by the alert generation module 206, an alert for a medical care supervisor through the EMR.

As will be also appreciated, the above described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, cloud storage, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

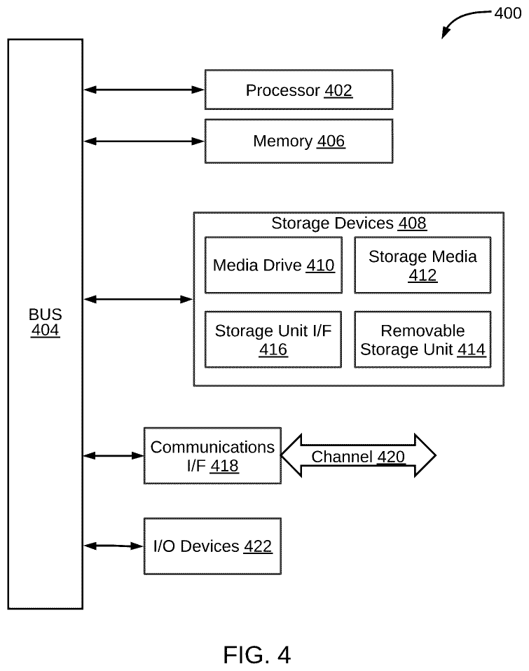

The disclosed methods and systems may be implemented on a conventional or a general-purpose computer system, such as a personal computer (PC) or server computer. Referring now to FIG. 4, an exemplary computing system 400 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 400 may represent, for example, a user device such as a desktop, a laptop, a mobile phone, personal entertainment device, DVR, and so on, or any other type of special or general-purpose computing device as may be desirable or appropriate for a given application or environment. The computing system 400 may include one or more processors, such as a processor 402 that may be implemented using a general or special purpose processing engine such as, for example, a microprocessor, microcontroller, or other control logic. In this example, the processor 402 is connected to a bus 404 or other communication medium. In some embodiments, the processor 402 may be an Artificial Intelligence (AI) processor, which may be implemented as a Tensor Processing Unit (TPU), or a graphical processor unit, or a custom programmable solution Field-Programmable Gate Array (FPGA).

The computing system 400 may also include a memory 406 (main memory), for example, Random Access Memory (RAM) or other dynamic memory, for storing information and instructions to be executed by the processor 402. The memory 406 also may be used for storing temporary variables or other intermediate information during execution of instructions to be executed by the processor 402. The computing system 400 may likewise include a read only memory (“ROM”) or other static storage device coupled to bus 404 for storing static information and instructions for the processor 402.

The computing system 400 may also include a storage device 408, which may include, for example, a media drive 410 and a removable storage interface. The media drive 410 may include a drive or other mechanism to support fixed or removable storage media, such as a hard disk drive, a floppy disk drive, a magnetic tape drive, an SD card port, a USB port, a micro USB, an optical disk drive, a CD or DVD drive (R or RW), or other removable or fixed media drive. A storage media 412 may include, for example, a hard disk, magnetic tape, flash drive, or other fixed or removable medium that is read by and written to by the media drive 410. As these examples illustrate, the storage media 412 may include a computer-readable storage medium having stored therein particular computer software or data.

In alternative embodiments, the storage devices 408 may include other similar instrumentalities for allowing computer programs or other instructions or data to be loaded into the computing system 400. Such instrumentalities may include, for example, a removable storage unit 414 and a storage unit interface 416, such as a program cartridge and cartridge interface, a removable memory (for example, a flash memory or other removable memory module) and memory slot, and other removable storage units and interfaces that allow software and data to be transferred from the removable storage unit 414 to the computing system 400.

The computing system 400 may also include a communications interface 418. The communications interface 418 may be used to allow software and data to be transferred between the computing system 400 and external devices. Examples of the communications interface 418 may include a network interface (such as an Ethernet or other NIC card), a communications port (such as for example, a USB port, a micro USB port), Near field Communication (NFC), etc. Software and data transferred via the communications interface 418 are in the form of signals which may be electronic, electromagnetic, optical, or other signals capable of being received by the communications interface 418. These signals are provided to the communications interface 418 via a channel 420. The channel 420 may carry signals and may be implemented using a wireless medium, wire or cable, fiber optics, or other communications medium. Some examples of the channel 420 may include a phone line, a cellular phone link, an RF link, a Bluetooth link, a network interface, a local or wide area network, and other communications channels.

The computing system 400 may further include Input/Output (I/O) devices 422. Examples may include, but are not limited to a display, keypad, microphone, audio speakers, vibrating motor, LED lights, etc. The I/O devices 422 may receive input from a user and also display an output of the computation performed by the processor 402. In this document, the terms “computer program product” and “computer-readable medium” may be used generally to refer to media such as, for example, the memory 406, the storage devices 408, the removable storage unit 414, or signal(s) on the channel 420. These and other forms of computer-readable media may be involved in providing one or more sequences of one or more instructions to the processor 402 for execution. Such instructions, generally referred to as “computer program code” (which may be grouped in the form of computer programs or other groupings), when executed, enable the computing system 400 to perform features or functions of embodiments of the present invention.

In an embodiment where the elements are implemented using software, the software may be stored in a computer-readable medium and loaded into the computing system 400 using, for example, the removable storage unit 414, the media drive 410 or the communications interface 418. The control logic (in this example, software instructions or computer program code), when executed by the processor 402, causes the processor 402 to perform the functions of the invention as described herein.

Thus, the disclosed method and system try to overcome the technical problem of misplacement and mispositioning of the medical lines and tubes in the patient body. The method and system allow for real-time monitoring of malplacement and malpositioning of the medical lines and tubes. The method and system facilitate a timely intervention in case of peri-procedural complications related to malplacement and malpositioning of the medical lines and tubes. Further, the method and system generate real-time alerts for the misplacement, mispositioning, and blockages of the medical lines and tubes. The method and system decrease chances of human error in critical settings of the medical lines and tubes.

As will be appreciated by those skilled in the art, the techniques described in the various embodiments discussed above are not routine, or conventional, or well understood in the art. The techniques discussed above provide for detecting malplacement and malpositioning of medical lines and tubes. The techniques first receive real-time image data corresponding to a patient from one or more cameras and patient data from an EMR of the patient. The techniques then determine a set of optimal parameters for each of the at least one medical line or tube with respect to a corresponding body part of the patient based on the patient data. The techniques then detect an malplacement and malposition corresponding to the at least one medical lines and tubes based on the real-time image data, the set of optimal parameters, and predefined insertion criteria using an ML model.

In light of the above mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has described the method and system for detecting malplacement and malpositioning of medical lines and tubes. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, cloud storage, and any other known physical storage media.

It is intended that the disclosure and examples be considered as exemplary only, with a true scope and spirit of disclosed embodiments being indicated by the following claims.

What is claimed is:

1. A method for detecting malplacement and malpositioning of medical lines or tubes, the method comprising:
receiving, by a detection device, real-time image data corresponding to a patient from one or more cameras and patient data from an Electronic Medical Record (EMR) of the patient, wherein at least one medical line or tube is at least partially inserted in at least one body part of the patient, and wherein the real-time image data comprises a plurality of images capturing the at least one medical line or tube and the corresponding at least one body part;
determining, by the detection device, a set of optimal parameters for each of the at least one medical line or tube with respect to a corresponding body part of the patient based on the patient data; and
detecting, by the detection device, malplacement and malpositioning of the at least one medical line or tube based on the real-time image data, the set of optimal parameters, and predefined insertion criteria using a Machine Learning (ML) model.

2. The method as claimed in claim 1, wherein each of the plurality of images of the real-time image data is one of a regular light image, an infrared (IR) light image, or a video frame.

3. The method as claimed in claim 1, comprising training the ML model based on a training dataset using supervised learning techniques.

4. The method as claimed in claim 1, wherein detecting the malplacement and malpositioning corresponding to the at least one medical line or tube comprises:
identifying the at least one medical line or tube in an image via object detection and boundary detection techniques using the ML model;
identifying a site of contact of each of the identified at least one medical line or tube with the corresponding body part of the patient using the ML model;
determining a set of current parameter values based on each of the identified at least one medical line or tube and the identified site of contact;
comparing the set of current parameter values with the corresponding set of optimal parameter values; and
detecting the malplacement and malpositioning based on the comparison and the predefined insertion criteria.

5. The method as claimed in claim 1, comprising classifying the at least one medical line or tube into a patient intake line or a patient output line using the ML model.

6. The method as claimed in claim 1, comprising, upon detecting the malplacement and malpositioning, generating an alert for a medical care supervisor through the EMR.

7. A system for detecting malplacement and malpositioning of medical lines or tubes, the system comprising:
a processor; and
a memory communicatively coupled to the processor, wherein the memory stores processor instructions, which when executed by the processor, cause the processor to:
receive real-time image data corresponding to a patient from one or more cameras and patient data from an Electronic Medical Record (EMR) of the patient, wherein at least one medical line or tube is at least partially inserted in at least one body part of the patient, and wherein the real-time image data comprises a plurality of images capturing the at least one medical line or tube and the corresponding at least one body part;
determine a set of optimal parameters for each of the at least one medical line or tube with respect to a corresponding body part of the patient based on the patient data; and
detect malplacement and malpositioning of the at least one medical line or tube based on the real-time image data, the set of optimal parameters, and predefined insertion criteria using a Machine Learning (ML) model.

8. The system as claimed in claim 7, wherein each of the plurality of images of the real-time image data is one of a regular light image, an infrared (IR) light image, or a video frame.

9. The system as claimed in claim 7, wherein the processor instructions, on execution, cause the processor to training the ML model based on a training dataset using supervised learning techniques.

10. The system as claimed in claim 7, wherein to detect the malplacement and malpositioning corresponding to the at least one medical line or tube, the processor instructions, on execution, cause the processor to:
identify the at least one medical line or tube in an image via object detection and boundary detection techniques using the ML model;
identify a site of contact of each of the identified at least one medical line or tube with the corresponding body part of the patient using the ML model;
determine a set of current parameter values based on each of the identified at least one medical line or tube and the identified site of contact;
compare the set of current parameter values with the corresponding set of optimal parameter values; and
detect the malplacement and malpositioning based on the comparison and the predefined insertion criteria.

11. The system as claimed in claim 7, wherein the processor instructions, on execution, cause the processor to classify the at least one medical line or tube into a patient intake line or a patient output line using the ML model.

12. The system as claimed in claim 7, wherein upon detecting the malplacement and malpositioning, the processor instructions, on execution, cause the processor to generate an alert for a medical care supervisor through the EMR.

13. A non-transitory computer-readable medium storing computer-executable instructions for detecting malplacement and malpositioning of medical lines or tubes, the computer-executable instructions configured for:
receiving real-time image data corresponding to a patient from one or more cameras and patient data from an Electronic Medical Record (EMR) of the patient, wherein at least one medical line or tube is at least partially inserted in at least one body part of the patient, and wherein the real-time image data comprises a plurality of images capturing the at least one medical line or tube and the corresponding at least one body part;
determining a set of optimal parameters for each of the at least one medical line or tube with respect to a corresponding body part of the patient based on the patient data; and
detecting malplacement and malpositioning of the at least one medical line or tube based on the real-time image data, the set of optimal parameters, and predefined insertion criteria using a Machine Learning (ML) model.

14. The non-transitory computer-readable medium as claimed in claim 13, wherein each of the plurality of images of the real-time image data is one of a regular light image, an infrared (IR) light image, or a video frame.

15. The non-transitory computer-readable medium as claimed in claim 13, wherein the computer-executable instructions are configured for training the ML model based on a training dataset using supervised learning techniques.

16. The non-transitory computer-readable medium as claimed in claim 13, wherein to detect the malplacement and malpositioning corresponding to the at least one medical line or tube, the computer-executable instructions are configured for:
identifying the at least one medical line or tube in an image via object detection and boundary detection techniques using the ML model;
identifying a site of contact of each of the identified at least one medical line or tube with the corresponding body part of the patient using the ML model;
determining a set of current parameter values based on each of the identified at least one medical line or tube and the identified site of contact;
comparing the set of current parameter values with the corresponding set of optimal parameter values; and
detecting the malplacement and malpositioning based on the comparison and the predefined insertion criteria.

17. The non-transitory computer-readable medium as claimed in claim 13, wherein the computer-executable instructions are configured for classifying the at least one medical line or tube into a patient intake line or a patient output line using the ML model.

18. The non-transitory computer-readable medium as claimed in claim 13, wherein the computer-executable instructions are configured for, upon detecting the malplacement and malpositioning, generating an alert for a medical care supervisor through the EMR.
